(19) United States
(12) Patent Application Publication
(10) Pub. No.: US 2016/0361119 A1
(43) Pub. Date: Dec. 15, 2016

(54) METHOD AND APPARATUS FOR CUTTING AND SHAVING HUMAN AND ANIMAL HAIRS AND NATURAL OR ARTIFICIAL FIBERS

(71) Applicant: UMM AL-QURA UNIVERSITY, Makkah (SA)

(72) Inventor: Fadel M. Y. OTHMAN, Makkah (SA)

(73) Assignee: UMM AL-QURA UNIVERSITY, Makkah (SA)

(21) Appl. No.: 14/736,056

(22) Filed: Jun. 10, 2015

(51) Int. Cl. A61B 18/20; A61B 18/22

(52) U.S. Cl. CPC A61B 18/203; A61B 18/22; A61B 2018/00601

(57) ABSTRACT

An apparatus is provided to cut hair using laser light. The apparatus may include a handle portion having a proximal end. The handle portion may include a power source and processing circuitry. The handle portion may also include a laser generator configured to generate a laser light configured to cut hair or fiber shafts. The laser generator is configured to generate laser light having a wavelength selected to target a predetermined chromophore to effectively cut a hair shaft. The apparatus may also include a comb-like portion having a distal end. The comb-like portion may include a heat sink portion conductively coupled to the laser generator. The heat sink portion includes a heat dissipation material configured to change between at least two phases.

### BACKGROUND

Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Shaving is the removal of hair, by using a razor or any other kind of bladed implement, to slice it down to the level of the skin or otherwise. Shaving is most commonly practiced by men to remove their facial hair and by women to remove their leg and underarm hair. A man is called clean-shaven if he has had his beard and mustache entirely removed.

Head shaving is much more common among men. It is often associated with religious practice, the armed forces, and among some competitive sports such as swimming, running and extreme sports. Existing razors made of metal or other materials need to be in direct contact with the skin to create shear force for shaving. Shaving and hair-cutting is a large industry. More efficient, hygiene, fast and easy to use apparatus is needed and appreciated.

In some occasions where a large number of people need to shave their heads in a short period of time, they may shave for themselves or for their friends. However, unskilled people may work as barbers due to the large demand and high profit margins.

Shaving can have or lead to numerous side effects, including cuts, abrasions, and irritation. The risk is increased if the shaving is done in a rush by unskilled people.

Straight razors consist of a blade sharpened on one edge. The blade can be made of either stainless steel, which is slower to hone and strop, and holds an edge longer, or high carbon steel, which hones and strops quickly, but has a less durable edge. Straight razors are quite dangerous in unexperienced hands as it may cause serious cuts.

Double sided safety razors are safer in terms of limited razor area exposed, but may cause skin burns especially if used in a rush. Both types of razors require wet lubricants or gel for shaving. Also, neither type of razor is suitable for large skin areas due to safety and infection problems and messy waste.

Further, cuts from shaving can bleed for about fifteen minutes or so, thereby increasing the risk of infection. Shaving cuts can be caused by blade movement perpendicular to the blade's cutting axis or by regular/orthogonal shaving over prominent bumps on the skin (which the blade incises).

Electric shavers consist of a set of oscillating or rotating blades, which are held behind a perforated metal screen which prevents them from coming into contact with the skin and behaves much like the second blade in a pair of scissors. When the razor is held against the skin, the whickers poke through the holes in the screen and are sliced by the moving blades. In some designs, the blades are a rotating cylinder. In other designs, they are one or more rotating disks or a set of oscillating blades. Such electric razors can be suitable for personal use, but not for public use due to hygiene and practical reasons. Electric razors also need frequent maintenance due to hair waste and blade wear.

Manual and electric hair cutters are quite fast and efficient, but require routine maintenance and lubrication. Further, these cutters merely cut hair but do not shave hair.

### SUMMARY

Embodiments include an apparatus configured to cut hair using laser light, the apparatus includes a handle portion having a proximal end. The handle portion includes a power source and processing circuitry. The handle portion also includes a laser generator configured to generate a laser light configured to cut hair or fiber shafts, the laser generator further configured to generate laser light having a wavelength selected to target a predetermined chromophore to cut a hair shaft. The apparatus also includes a comb-like portion having a distal end. The comb-like portion includes a heat sink portion conductively coupled to the laser generator. The heat sink portion includes a heat dissipation material configured to change between at least two phases.

Embodiments include a method which includes providing a laser cutting apparatus configured to cut hair shafts or fibers. The method also includes cutting hair shafts or fiber proximal a user's skin or scalp via a laser beam. The method further includes comparing the cut hair shafts or fiber to a predetermined chromophore. The method also includes reflecting the laser beam into an interior space of the laser cutting apparatus. The method further includes absorbing the reflected laser beam via a heat sink and a phase changing material, wherein the heat sink includes a heat dissipation material configured to change between at least two phases.

Embodiments include an apparatus including means for cutting hair shafts or fiber proximal a user's skin or scalp via a laser beam. The apparatus also includes means for comparing the cut hair shafts or fiber to a predetermined chromophore. The apparatus further includes means for reflecting the laser beam into an interior space of the laser cutting apparatus. The apparatus also includes means for absorbing the reflected laser beam via a heat sink and a phase changing material.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an illustrative view of a laser razor apparatus according to certain embodiments of the disclosure.

FIG. 2 is an illustrative view of the laser razor apparatus of FIG. 1 according to certain embodiments of the disclosure.

FIG. 3A is a close-up illustrative view of the laser razor apparatus of FIG. 1 according to certain embodiments of the disclosure.

FIG. 3B is a diagrammatic view of the laser razor apparatus according to certain embodiments of the disclosure.

FIG. 4 is a diagrammatic view of an end tooth having a laser reflector of the laser razor apparatus according to certain embodiments of the disclosure.

FIG. 5 is a schematic view of an end tooth having a laser reflector of the laser razor apparatus according to certain embodiments of the disclosure.

FIG. 6 is an illustrative view of a removable cutting guide for the laser razor apparatus according to certain embodiments of the disclosure.

FIG. 7 is a flowchart illustrating a method of cutting according to certain embodiments of the disclosure.

FIG. 8 is a block diagram of a controller for the apparatus of FIG. 1 according to certain embodiments of the disclosure.

### DETAILED DESCRIPTION OF THE EMBODIMENTS

Referring now to the drawings, wherein like reference numerals designate identical or corresponding parts throughout the several views.

The present disclosure is related to devices used to cut or shave human and animal hairs, and unwanted fibers scattered from fabrics.

Razors made of metal or other materials are well established for decades. However, a laser razor is a new concept where no sharp blade is needed, since cutting action is done by burning a small portion of the hair. Powerful laser beams are currently used for cutting sheets of metal or other materials, or as a cutter for surgeries. None of these laser beams is employed to function as a razor blade for shaving hair. Current hair treatments by laser are mainly for permanently removing unwanted hair from some part of the body by killing the roots of the hair. The present disclosure provides a method and apparatus which cuts the hair just above the skin, thereby leaving the roots unaffected.

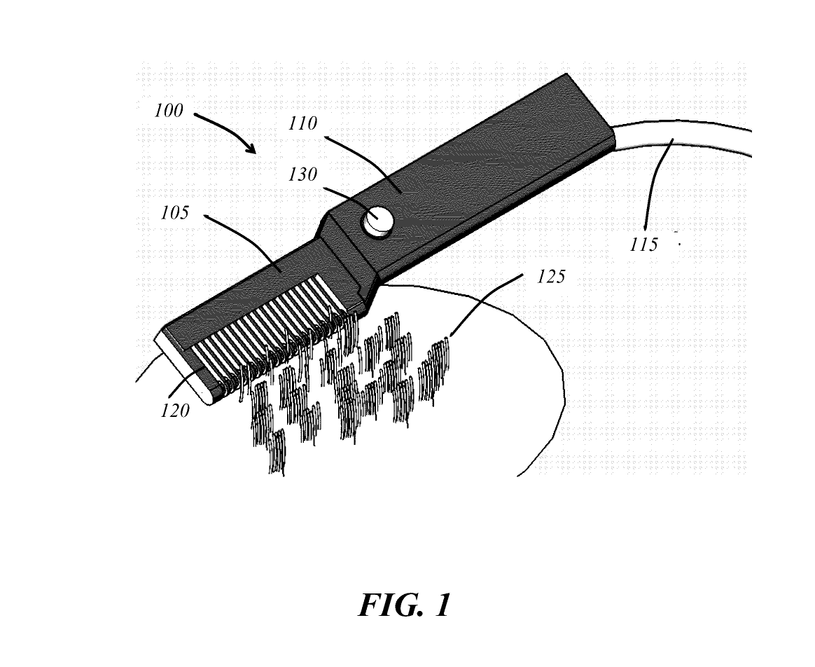

FIG. 1 is an illustrative view of a laser razor apparatus 100 according to certain embodiments of the disclosure. In FIG. 1, apparatus 100 may include a comb-like portion 105, a handle portion 110 disposed at a proximal end of the comb-like portion 105, a fiber optic 115, and an end teeth portion 120 disposed at a distal end of the comb-like portion 105.

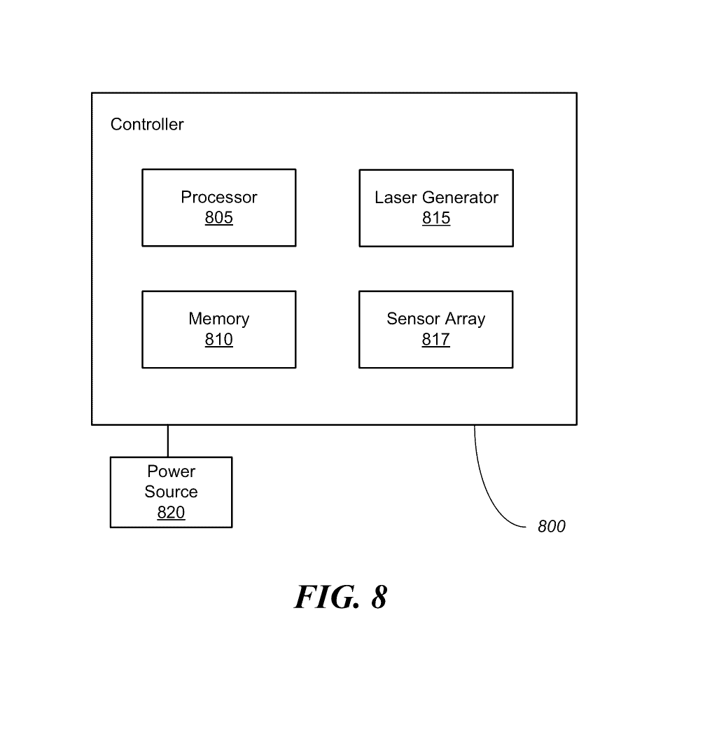

Handle portion 110 may include a controller 800 having at least one processor 805, at least one memory 810, a laser generator 815, and a power source 820 (see FIG. 8). Fiber optic 115 may be coupled to an external laser source (not shown) having its own power source. Fiber optic 115 may extend from a proximal end of handle portion 110 to the distal end teeth portion 120 of comb-like portion 105. Further, fiber optic 115 may be configured as a waveguide for a light beam 300 configured to cut hairs. Distal end teeth portion 120 may be integral with comb-like portion 105 and configured to engage a user's skin/scalp area 125 during a laser cutting operation.

Apparatus 100 is configured to shave or cut hair shafts on the user's skin/scalp area 125. Apparatus 100 may also include a safety button 130 configured to manually activate and de-activate laser generator 815 or fiber optic transmission when pressed down by the user.

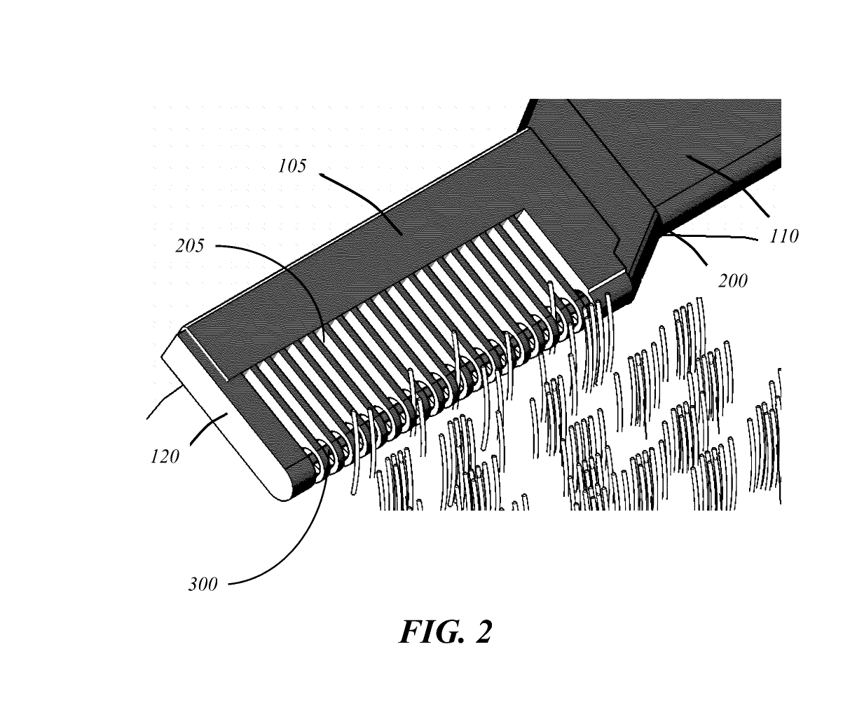

FIG. 2 is an illustrative view of the laser razor apparatus 100 according to certain embodiments of the disclosure. In FIG. 2, apparatus 100 may include an angled bend portion 200 having laser beam reflectors (not shown) disposed therein and configured to direct the placement of a light beam 300 within comb-like portion 105. Apparatus 100 may also include a plurality of tooth spacing 205 disposed proximal end tooth portion 120. Tooth spacings 205 may be configured to allow hair or fiber shafts to pass there through during the use of apparatus 100.

In certain embodiments, light beam 300 may utilize a laser medium Neodymium (3+) doped, Yttrium-Aluminum Garnet (Nd:YAG), with a peak absorption occurring at about 808 nanometers (nm). Also, other laser mediums such as Nd:YLF can be used. Further, light beam 300 is configured to easily cut human or animal hair shafts while using a wavelength of about 380 to 480 nm, 380 to 500 nm, 2500 to 3500 nm, 2950 to 3050 nm, and/or 2700 to 3500 nm. In any embodiment according to the present disclosure, the light beam 300 can be laser light, coherent light, and/or at least one part of non-collimated light. These wavelengths may be selected to target a predetermined chromophore to effectively cut a hair shaft via light beam 300. To damage and/or cut one or more hair shafts with light, at least some of the light energy is absorbed by the hair shaft and converted into heat or induce a bond breaking mechanism. There are three chromophores in hair that substantially absorb light, namely, melanin, keratin, and water. Keratin and water have absorption peaks at around 3000 nm. Melanin has an absorption peak around 300 nm, but remains relatively flat, decreasing almost linearly (on a logarithmic scale) to about 3000 nm. Further, chromophores may chemically include sebum, a fatty acid, phytoshingosine, ceramide, cholesterol, cholesterol sulfate, and cholesterol oleate. In some such embodiments, the light passes directly from the light shaping optic to the hair. In other embodiments, the light passes through at least one more optic to be directed to the hair.

In some embodiments, apparatus 100 includes a light shaping optic. The light shaping optic is a waveguide or fiber that at least partially changes the shape of light emitted to a line having a length of between about 2 mm and about 250 mm. In some embodiments, light from the laser generator 815 is coupled into at least one blade shaped optic that guides at least part of the light to the hair. The blade shaped optic can be a light guide and/or a light transmitter. The blade shaped optic can be detachable, consumable, and/or exchangeable.

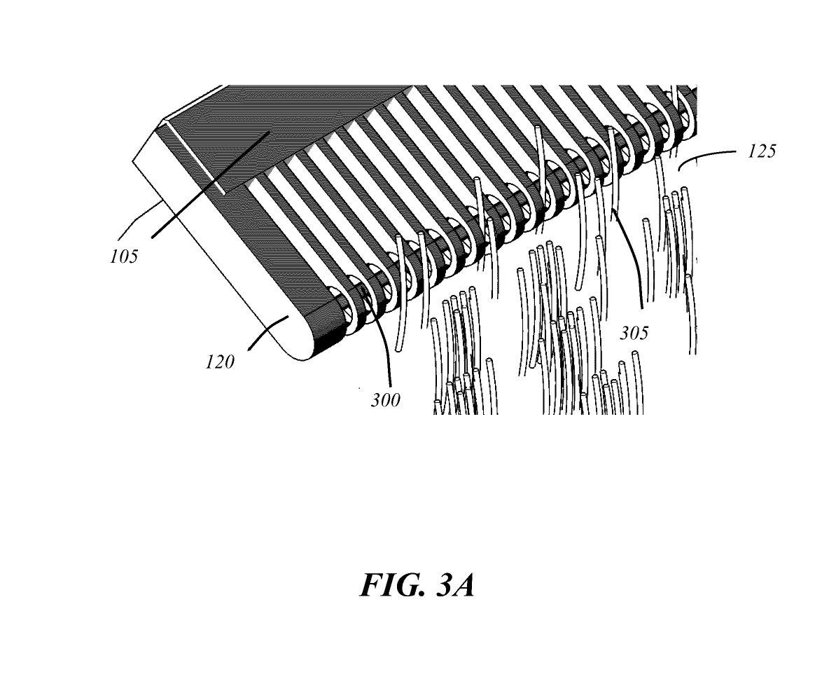

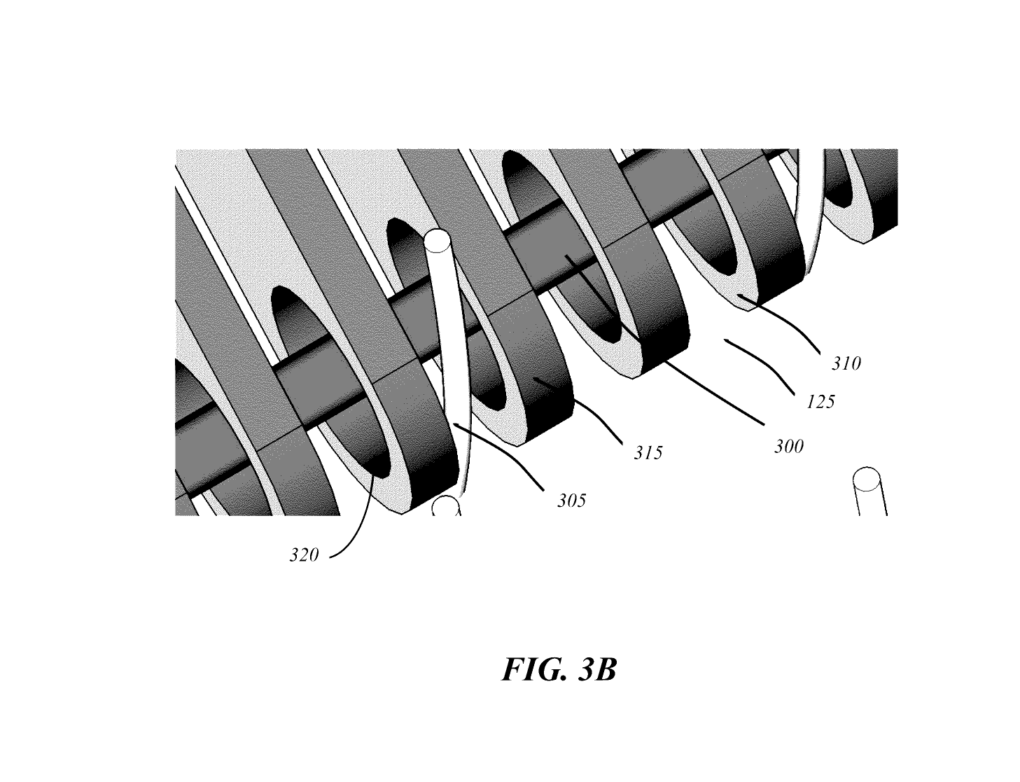

FIG. 3A is a close-up illustrative view of the laser razor apparatus 100 of FIG. 2 according to certain embodiments of the disclosure. In FIG. 3A, apparatus 100 is shown with light beam 300 activated to cut hair shafts 305 upon contact. The cut as shown is configured to cut perpendicular to hair shafts 305 and spaced apart from the user's skin/scalp area 125 for safety purposes.

FIG. 3B is a diagrammatic view of the laser razor apparatus 100 according to certain embodiments of the disclosure. In FIG. 3B, apparatus 100 is shown with light beam 300 activated to cut hair shafts 305. In apparatus 100, spacer rings 310 are configured to protect the user's skin/scalp area 125 as light beam 300 passes there through during use. Spacer rings 310 may have a circumferential thickness of about 0.5 to 1.5 millimeters (mm) and are disposed proximal end teeth 315.

In some embodiments, end teeth portion 120 may be configured as movable teeth which float within comb-like portion 105 on pistons or springs in order to allow for the end teeth portion 120 to map the contours of the user's skin/scalp area 125 during use of apparatus 100 while maintaining spacer rings 310 to protect the user from the light beam 300. End teeth portion 120 may further be configured to include circular openings or channel 320 disposed in each tooth of end teeth portion 120 at a distal end thereof. Light beam 300 is configured to be emitted along a central axis of channel 320.

In either embodiment, hair shafts 305 pass between end teeth portion 120 of apparatus 100 and the lower part of hair shafts 305 is burned by light beam 300 effecting a shave or cut. Further, end teeth portion 120 may be comprised of an antibacterial material, such as black silicon. Under a microscope the surface of black silicon includes a forest of spikes just 500 nanometers (nm) high that rip open the cell walls of any bacterium which comes into contact with it. These regularly-spaced “nanopillars” slice bacteria to shreds as the bacteria settle on the surface. Further, these surfaces are smooth to human touch.

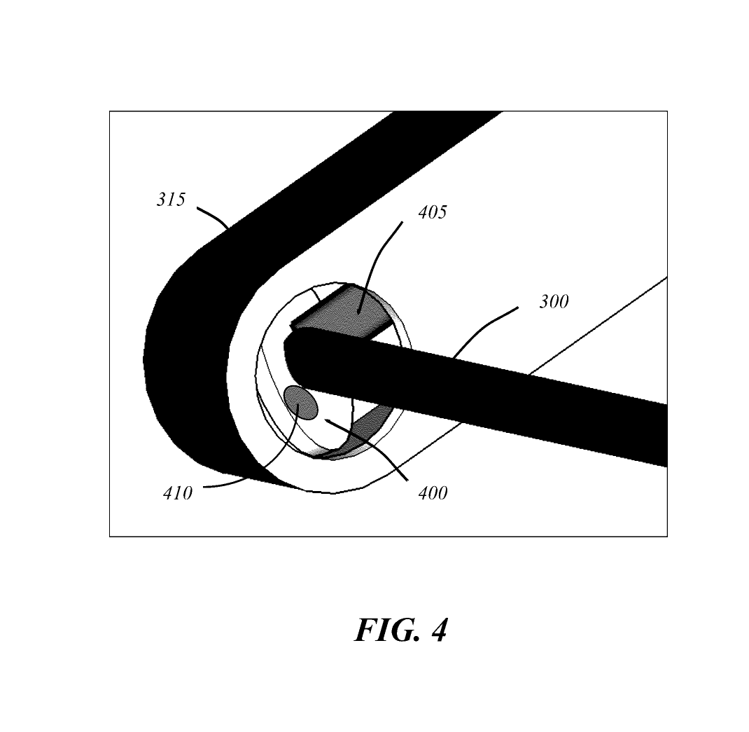

FIG. 4 is a diagrammatic view of an end tooth 315 having a laser reflector 400 of the laser razor apparatus 100 according to certain embodiments of the disclosure. In FIG. 4, an end tooth 315 of apparatus 100 includes a reflector 400 configured to reflect the incident light beam 300 and redirect it as a reflected beam 405 into an inner body extinction space 500 within apparatus 100 for heat dissipation and cooling. In some embodiments, reflector 400 has a reflectivity of about 75 to 94%. In other embodiments, reflector 400 may include an optic sensor 410 disposed proximal the circumference of the reflector 400. Optic sensor 410 may be configured to detect cut hairs or fibers in order to provide inputs to the processing circuitry which may be configured to make a comparison of the detected cuts to that of a predetermined cut profile saved in a memory of a controller 800 having processing circuitry and a power source 820 (see FIG. 8).

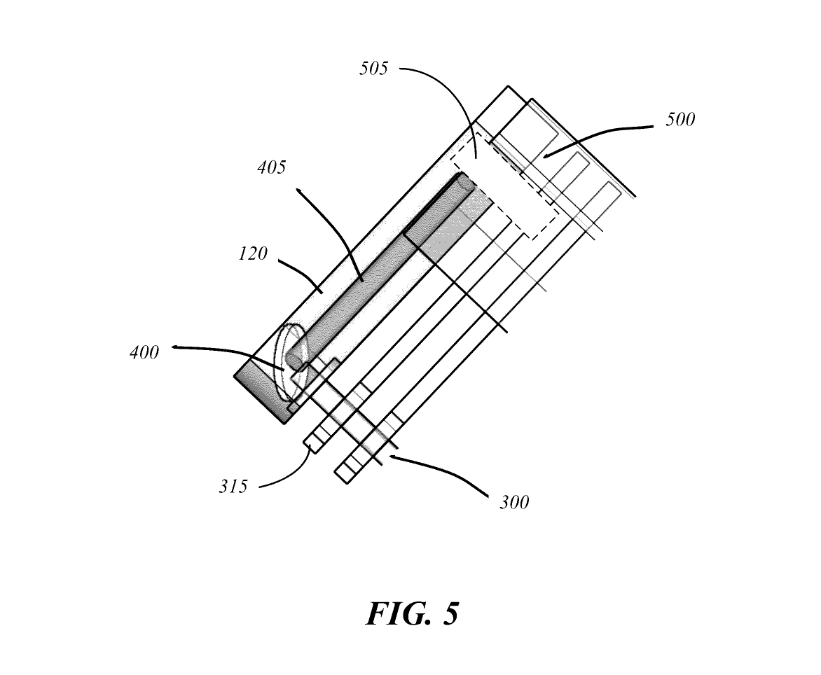

FIG. 5 is a schematic view of an end tooth 315 having a laser reflector 400 of the laser razor apparatus 100 according to certain embodiments of the disclosure. In FIG. 5, incident light beam 300 is redirected by reflector 400 to inner body extinction space 500 of apparatus 100 where reflected beam 405 is absorbed by a phase change material (PCM) 505 placed within space 500 and configured to change from a solid to a liquid form and in some embodiments into a gaseous form based on the energy rating of the light beam 300. A PCM 505 may be selected that has a melting point above the maximum ambient temperature of the environment in which apparatus 100 resides so that PCM will remain in its solid phase before laser 300 operation begins. This temperature is at or about −35 to 55° C. Other factors include the desired laser power output, size of both the laser medium and the laser diode array, and the efficiency of the laser diodes and laser medium which is proportional to the waste heat. PCM 505 may be comprised of gallium which has a melting point of 29.8° C. and a latent heat of fusion of 80 J/g. In some embodiments, PCM 505 may be include or be coupled to a vibration mechanism configured to remove PCM 505 when in liquid form from apparatus 100 after a phase change has occurred.

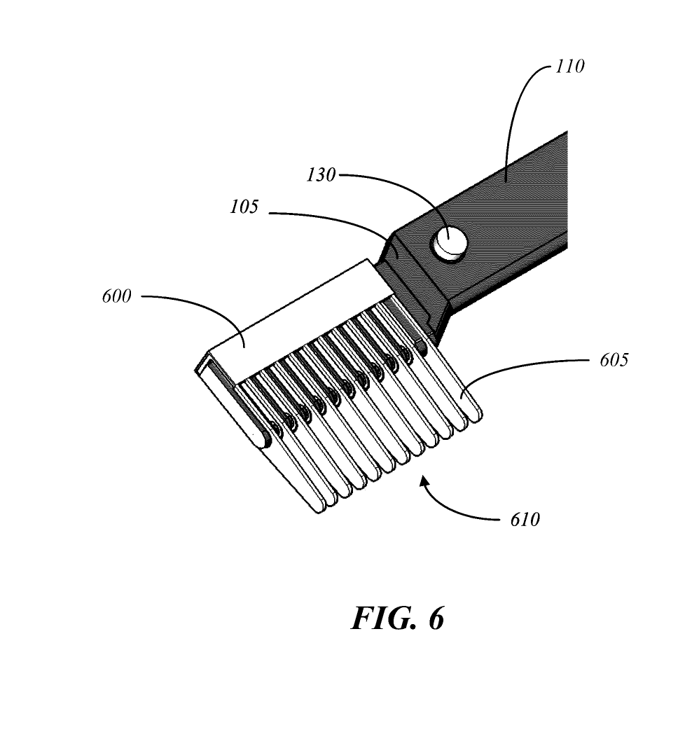

FIG. 6 is an illustrative view of a removable cutting guide 600 for the laser razor apparatus 100 according to certain embodiments of the disclosure. In FIG. 6, apparatus 100 may be configured to support or hold removable cutting guides, such as cutting guide 600 shown. Cutting guide 600 may be configured as an extension for cutting a user's head of hair, beard, or mustache, etc. Guide 600 may be part of a set of guides configured to cut different lengths of hair based on the user's preference or needs. These lengths may be fractions of an inch in the case of a beard or mustache to an inch or more to cut the user's head of hair as desired.

Guide 600 may include a plurality of comb teeth 605 with rounded distal tips/ends 610 configured to be incident with the user's hair/scalp area 125 during use. Guide 600 is also configured to act as a spacer between the light beam 300 and the user's hair/scalp area 125.

An apparatus 100 uses a narrow light beam 300 powerful enough for shaving or cutting hairs or fibers 305. It consists of a comb-like structure 105, where its teeth 310 have holes to allow laser beam to pass through, prevent it from touching the skin or fabric it shaves, and absorbs scattered rays to protect eyes.

The end tooth 120 is hollow where light beam 300, after cutting hairs, is reflected by a reflector 400, to the inner material of the device for heat dissipation.

Laser beam can be produced by a separate laser generator device and transmitted to the apparatus 100 through a suitable media such as fiber optics 115, or produced inside the handle 110 of the apparatus 100. Safety bottom 130 must be pressed to switch laser on. Extinction 500 can be attached to the apparatus 100 for the required length for haircut.

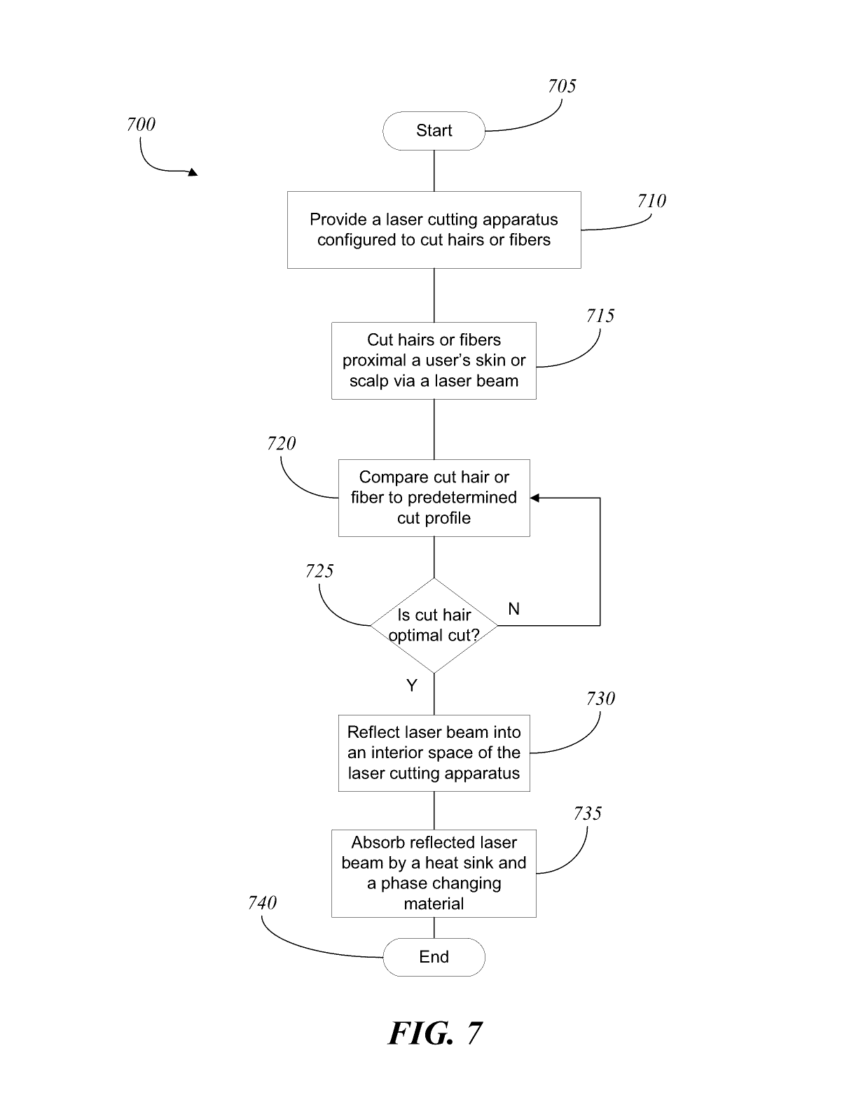

FIG. 7 is a flowchart illustrating a method 700 of cutting according to certain embodiments of the disclosure. In FIG. 7 at 705, apparatus 100 is activated or started. At 710, apparatus 100 is provided to cut hairs or fibers of a user. At 715, apparatus 100 cuts the hairs or fibers proximal a user's skin or scalp area via light beam 300. At 720, the cut hair or fiber is compared to a predetermined cut profile stored in processing circuitry within apparatus 100. This comparison is implemented via an optical sensor disposed proximal the light beam 300 cutting area. At 725, a condition is determined via the stored cut profile. At 730, light beam 300 is reflected into an interior space of the apparatus 100 for heat dissipation. At 735, the reflected light beam 300 is absorbed by a heat sink and a phase changing material. At 740, apparatus 100 is deactivated and the laser beam is turned off once the optimal hair or fiber cut is completed.

FIG. 8 is a block diagram of a controller 800 for apparatus 100 of FIG. 1 according to certain embodiments of the disclosure. In FIG. 8, controller 800 may disposed within apparatus 100 and include at least one processor 805, at least one memory 810, a laser generator 815, and a power source 820. Processor 805 may include processing circuitry configured to execute program code and the like. Memory 810 may include stored hair cut profiles for comparison to actual hair or fiber cuts performed by apparatus 100. Laser generator 815 may include laser medium and circuitry to create, control and maintain light beam 300 during use. Further, as discussed above, optical sensor 410 may be electrically connected to controller 800 to provide sensory input. Power source 820 may include a DC battery or AC power electrically connected to controller 800 and apparatus 100.

When cutting white or light hair with blue light, e.g., at about 403 nm, approximately twice the energy level is needed compared to cutting brown hair, for example, by targeting melanin. In some embodiments, an apparatus 100 as described herein may include one or more sensors 410 disposed in a sensor array 817 and configured to detect or gather data indicative of the chromophore(s) present in the target hair. For example, upon contact with the hair, the apparatus 100 can emit light into the hair, and the sensor 815 can detect the light reflected to allow the processing circuitry of the controller 800 to determine the wavelengths of light absorbed. In some embodiments, the sensor 815 could be located in the handle portion 110 of the apparatus 100. In some embodiments, the sensor 410 can be a MEMS device that functions as a spectrometer and is located on the portion of the apparatus 100 configured to emit light to and/or contact the hair. If the sensor 815 detects and/or the controller 800 determines based on sensor data that the hair contains a sufficient amount of melanin, the controller 800 can reduce the energy level or power and/or adjust the wavelength of light emitted to target a predetermined chromophore, such as melanin. If the sensor 815 detects and/or the controller 800 determines based on sensor data that the hair lacks sufficient melanin but contains sufficient sebum, the controller 800 can increase the energy level or power and/or adjust the wavelength emitted to target the sebum.

More than one apparatus 100 as described herein can be used synchronously or in sequence to cut or damage hair.

Although the devices and methods herein have been described with respect to cutting or damaging hair, these devices and methods can be used for other applications, for example, surgery. The comb-like portion 105 can be shaped similar to a knife, surgical scalpel, or other cutting tool. In some embodiments, when using the apparatus 100 to cut tissue, the apparatus 100 can also act as a coagulating and bleeding stopping means by means of the heat created by light absorbed into the tissue. The light can be modulated and tuned to cut tissue or coagulate.

Apparatus 100 may provide many shaving advantage, such as a high level of hygiene being achieved since a laser beam shaves hair without skin cuts. This configuration may prevent infections and bleeding during shaving. Further, apparatus 100 may have low maintenance requirements, i.e., no mechanical parts need to be cleaned, lubricated, or replaced as with conventional shavers. Also, using apparatus 100 results in a fast and effortless process. In other words, the laser cuts hair at high speed and no force against the hair is required. In addition, apparatus 100 is used in a dry process that does not require any foam, shampoo or lubricant liquids to softening hair and skin.

Some additional advantages of apparatus 100 may include companies that produce shaving and haircut equipment are most likely interested in developing and producing such apparatus, especially for barber shops market and medical equipment manufacturers may also be interested as shaving hair is required for some surgeries and apparatus 100 is expected to make very clean cut with least possibilities for infection.

Some regulatory bodies and customers may insist to use this laser blades for hygiene reasons to avoid the problems of infections and cuts associated with normal shaving blades.

In large scale events, especially in the Hajj (the Moslem annual pilgrimage to Makkah) where numerous numbers of people shave their scalp, apparatus 100 may prove very useful for hygiene and practical reasons.

Further, apparatus 100 solves the problems of contamination, infection, and blood transmitted diseases associated by using normal blades. For instance, conventional metal blades irritate skin while pushing hair to cut it causing skin burns. Accidental movement of blades causes skin cuts. Laser blade solves these problems as it is not in direct contact with the skin. Also, using conventional metal blades are tedious and messy, as they requires continues force to cut hair and using lubricants for hair and skin. Light beam 300 of apparatus 100 is fast, dry, and less effort process, as light beam 300 cuts hair at high speed with no shear force or lubricants being required.

Thus, apparatus 100 provides the benefits of preventing contamination or blood transmitted diseases, preventing skin cuts or irritations, provides a dry, fast and almost effortless process, and there are no supplies required for hair and skin preparation for shaving, such as pre-electric balm, shaving cream and/or shaving gel.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

The above disclosure also encompasses the embodiment noted below.

(1) An apparatus configured to cut hair using laser light, the apparatus comprising: a handle portion having a proximal end, the handle portion comprising a power source; processing circuitry; and a laser generator configured to generate a laser light configured to cut hair or fiber shafts, the laser generator further configured to generate laser light having a wavelength selected to target a predetermined chromophore to cut a hair shaft; and a comb-like portion having a distal end, the comb-like portion comprising a heat sink portion conductively coupled to the laser generator, wherein the heat sink portion includes a heat dissipation material configured to change between at least two phases.

(2) The apparatus according to (1), wherein the heat sink portion includes a vibration mechanism configured to remove the phase changed material from the comb-like portion during heat dissipation.

(3) The apparatus according to (1) or (2), further comprising: a guide portion disposed in the comb-like portion and configured to space apart the emitted light beam from a skin of a user, and wherein the guide portion is configured to include spacing elements.

(4) The apparatus according to any one of (1) to (3), wherein the processing circuitry is configured to adjust an intensity of the laser generator, where the adjustable intensity of the laser generator is determined by a profile of hair characteristics stored in the processing circuitry.

(5) The apparatus according to any one of (1) to (4), further comprising: an optical sensor controlled by the processing circuitry, where the optical sensor is configured to view and compare cut hair or fiber shafts to a predetermined cut profile to optimize the cut.

(6) The apparatus according to any one of (1) to (5), wherein the handle portion is coupled to a fiber optic waveguide configured to transmit the light beam through the comb-like portion from the handle portion, where the fiber optic has a proximal end, a distal end, an outer wall, and a cutting region positioned towards the distal end and extending through the comb-like portion.

(7) The apparatus according to any one of (1) to (6), further comprising: a sensor array coupled to the processing circuitry and disposed in the comb-like portion proximal the emitted light beam, wherein the sensor array is configured to detect chromophores.

(8) The apparatus according to any one of (1) to (7), wherein the fiber optic is configured to receive the light beam from the laser generator at the proximal end of the handle portion, conduct the laser light from the proximal end of the handle portion to the distal end of the comb-like portion.

(9) The apparatus according to any one of (1) to (8), wherein the predetermined chromophore is selected from the group consisting of: sebum, a fatty acid, phytoshingosine, ceramide, cholesterol, cholesterol sulfate, and cholesterol oleate.

(10) The apparatus according to any one of (1) to (9), wherein the adjustable intensity of the laser generator is a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

(11) A method, comprising: providing a laser cutting apparatus configured to cut hair shafts or fibers; cutting hair shafts or fiber proximal a user's skin or scalp via a laser beam; comparing the cut hair shafts or fiber to a predetermined chromophore; reflecting the laser beam into an interior space of the laser cutting apparatus; and absorbing the reflected laser beam via a heat sink and a phase changing material, wherein the heat sink includes a heat dissipation material configured to change between at least two phases.

(12) The method according to (11), wherein the laser beam is adjustable with a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

(13) The method according to (11) or (12), wherein the predetermined chromophore is selected from the group consisting of: sebum, a fatty acid, phytoshingosine, ceramide, cholesterol, cholesterol sulfate, and cholesterol oleate.

(14) The method according to any one of (11) to (13), wherein the heat sink includes a vibration mechanism configured to remove the phase changed material from the cutting apparatus during heat dissipation.

(15) The method according to any one of (11) to (14), further comprising: detecting the predetermined chromophore via a sensor array.

(16) An apparatus, comprising: means for cutting hair shafts or fiber proximal a user's skin or scalp via a laser beam; means for comparing the cut hair shafts or fiber to a predetermined chromophore; means for reflecting the laser beam into an interior space of the laser cutting apparatus; and means for absorbing the reflected laser beam via a heat sink and a phase changing material.

(17) The apparatus according to (16), wherein the laser beam is adjustable with a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

(18) The apparatus according to (16) or (17), wherein the predetermined chromophore is selected from the group consisting of: sebum, a fatty acid, phytoshingosine, ceramide, cholesterol, cholesterol sulfate, and cholesterol oleate.

1. An apparatus configured to cut hair using laser light, the apparatus comprising:
a handle portion having a proximal end, the handle portion comprising
a power source;
processing circuitry; and
a laser generator configured to generate a laser light configured to cut hair or fiber shafts, the laser generator further configured to generate laser light having a wavelength selected to target a predetermined chromophore to cut a hair shaft; and
a comb-like portion having a distal end, the comb-like portion comprising
a heat sink portion conductively coupled to the laser generator,
wherein the heat sink portion includes a heat dissipation material configured to change between at least two phases.

2. The apparatus according to claim 1, wherein the heat sink portion includes a vibration mechanism configured to remove the phase changed material from the comb-like portion during heat dissipation.

3. The apparatus according to claim 1, further comprising:
a guide portion disposed in the comb-like portion and configured to space apart the emitted light beam from a skin of a user, and
wherein the guide portion is configured to include spacing elements.

4. The apparatus according to claim 1, wherein the processing circuitry is configured to adjust an intensity of the laser generator, where the adjustable intensity of the laser generator is determined by a profile of hair characteristics stored in the processing circuitry.

5. The apparatus according to claim 1, further comprising:
an optical sensor controlled by the processing circuitry, where the optical sensor is configured to view and compare cut hair or fiber shafts to a predetermined cut profile to optimize the cut.

6. The apparatus according to claim 1, wherein the handle portion is coupled to a fiber optic waveguide configured to transmit the light beam through the comb-like portion from the handle portion, where the fiber optic has a proximal end, a distal end, an outer wall, and a cutting region positioned towards the distal end and extending through the comb-like portion.

7. The apparatus according to claim 1, further comprising:
a sensor array coupled to the processing circuitry and disposed in the comb-like portion proximal the emitted light beam,
wherein the sensor array is configured to detect chromophores.

8. The apparatus according to claim 6, wherein the fiber optic is configured to receive the light beam from the laser generator at the proximal end of the handle portion, conduct the laser light from the proximal end of the handle portion to the distal end of the comb-like portion.

9. The apparatus according to claim 1, wherein the predetermined chromophore is selected from the group consisting of: sebum, a fatty acid, phytoshingosine, ceramide, cholesterol, cholesterol sulfate, and cholesterol oleate.

10. The apparatus according to claim 4, wherein the adjustable intensity of the laser generator is a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

11. A method, comprising:
providing a laser cutting apparatus configured to cut hair shafts or fibers;
cutting hair shafts or fiber proximal a user's skin or scalp via a laser beam;
comparing the cut hair shafts or fiber to a predetermined chromophore;
reflecting the laser beam into an interior space of the laser cutting apparatus; and
absorbing the reflected laser beam via a heat sink and a phase changing material, wherein the heat sink includes a heat dissipation material configured to change between at least two phases.

12. The method according to claim 11, wherein the laser beam is adjustable with a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

13. The method according to claim 11, wherein the predetermined chromophore is selected from the group consisting of: sebum, a fatty acid, phytoshingosine, ceramide, cholesterol, cholesterol sulfate, and cholesterol oleate.

14. The method according to claim 11, wherein the heat sink includes a vibration mechanism configured to remove the phase changed material from the cutting apparatus during heat dissipation.

15. The method according to claim 11, further comprising:
detecting the predetermined chromophore via a sensor array.

16. An apparatus, comprising:
means for cutting hair shafts or fiber proximal a user's skin or scalp via a laser beam;
means for comparing the cut hair shafts or fiber to a predetermined chromophore;
means for reflecting the laser beam into an interior space of the laser cutting apparatus; and
means for absorbing the reflected laser beam via a heat sink and a phase changing material.

17. The apparatus according to claim 16, wherein the laser beam is adjustable with a wavelength within one or more ranges selected from a group consisting of 380 nm to 480 nm, 380 nm to 500 nm, 400 nm to 500 nm, 2500 nm to 3500 nm, 2950 nm to 3050 nm, and 2700 nm to 3500 nm.

18. The apparatus according to claim 16, wherein the predetermined chromophore is selected from the group consisting of: sebum, a fatty acid, phytoshingosine, ceramide, cholesterol, cholesterol sulfate, and cholesterol oleate.
